(19) United States
(12) Patent Application Publication
(10) Pub. No.: US 2024/0082125 A1
(43) Pub. Date: Mar. 14, 2024

(54) USE OF A SHORT CHAIN FATTY ACID AS ANTIDANDRUFF AGENT

(71) Applicants: NANYANG TECHNOLOGICAL UNIVERSITY, Singapore (SG); THE NATIONAL UNIVERSITY OF SINGAPORE, Singapore (SG); L'OEAL, Paris (FR)

(72) Inventors: Tarun CHOPRA, Singapore (SG); Viduthalai Rasheedkhan REGINA, Singapore (SG); Scott RICE, Singapore (SG)

(21) Appl. No.: 18/258,091

(22) PCT Filed: Dec. 20, 2021
(86) PCT No.: PCT/EP2021/086855

§ 371 Date: Jun. 30, 2023

(30) Foreign Application Priority Data
Dec. 21, 2020 (SG) 10202012868V
Jun. 22, 2021 (FR) 2106639

(51) Int. Cl. A61K 8/36; A61Q 5/00; A61Q 17/00; A61Q 19/00

(52) U.S. Cl. CPC A61K 8/36; A61Q 5/006; A61Q 17/005; A61Q 19/00

(57) ABSTRACT

The present invention concerns the cosmetic field, and especially cosmetic uses of at least one short chain fatty acid chosen from propionic acid, butyric acid, valeric acid, non-metallic salts thereof, esters thereof and mixtures thereof, or a conditioned culture medium obtained from at least one microorganism which is able to produce such short chain fatty acid(s), as antidandruff agent, for preventing and/or treating desquamative disorders of the skin associated with the excessive proliferation of yeasts of the Malassezia genus on the skin and for maintaining and/or restoring at a normal level, the ecoflora of the skin and especially by preventing excessive colonization of the skin by Malassezia genus and/or by mediating growth of Cutibacterium acnes.

The present invention concerns the cosmetic field, and especially cosmetic uses of at least one short chain fatty acid chosen from propionic acid, butyric acid, valeric acid, non-metallic salts thereof, esters thereof and mixtures thereof, or a conditioned culture medium obtained from at least one microorganism, which is able to produce such short chain fatty acid(s), as antidandruff agent, for preventing and/or treating desquamative disorders of the skin associated with the excessive proliferation of yeasts of the Malassezia genus on the skin and for maintaining and/or restoring at a normal level, the ecoflora of the skin and especially by preventing excessive colonization of the skin by the yeasts of the Malassezia genus and/or by mediating the growth of Cutibacterium acnes.

Desquamative disorders of the skin such as dandruff or seborrheic dermatitis affect up to 50% of the worldwide population. They affect both men and women and are perceived as having a very negative psychosocial impact. The appearance of dandruff is disagreeable both aesthetically and because of the discomfort it causes (especially tingling or itching), and as such many people confronted with this problem wish to eliminate it efficiently and permanently.

These disorders correspond to excessive and visible desquamation of the skin resulting from excessively rapid multiplication of the epidermal cells and their abnormal maturation. This phenomenon may be caused especially by excessively aggressive skin or hair treatments, extreme climatic conditions, stress, diet, fatigue and pollution. Dandruff and seborrheic dermatitis conditions usually result from a disorder of the skin microflora and more particularly from excessive colonization by a fungus which belongs to the family of yeasts of the Malassezia genus, especially Malassezia restricta species, and a lower abundance of Cutibacterium acnes, compared to a healthy scalp.

Many treatments have been developed with the principal objective of eradicating Malassezia yeasts from the skin. Thus, the activity of the active agents currently used, such as zinc pyrithione, piroctone olamine or selenium disulfide, is based mainly on their fungicidal property. However, it is well known, that many of these conventional agents extend their antimicrobial effect on at least one of the other bacteria and are therefore not selective to yeasts of the Malassezia genus, and especially Malassezia restricta species, which can therefore kill or impair the beneficial skin commensal microflora.

Thus, solutions to find novel active agents which have a selective growth inhibition activity against yeasts of the Malassezia genus (known for being responsible of skin desquamative disorders), and especially Malassezia restricta species has become a major challenge in order to keep and/or restore a healthy skin microflora and therefore respond to the needs of consumers.

An aim of the present invention is to provide an active agent that is effective for inhibiting the growth of the yeasts of the Malassezia genus (responsible for desquamative disorders of the skin), especially Malassezia restricta species, without extending its antimicrobial effect to other bacteria, in particular Staphylococcus epidermidis, and/or Staphylococcus capitis, and/or Cutibacterium acnes, which altogether constitute a large portion of the skin microbiome.

Another aim of the invention is to propose an active agent that can maintain and/or restore at a normal level, the ecoflora of the skin and especially by preventing excessive colonization of the skin by the yeasts of the Malassezia genus and/or by mediating the growth of Cutibacterium acnes.

The Applicant has discovered surprisingly that the cosmetic use of at least one short chain fatty acid having a chain length comprising no more than 5 carbon atoms chosen from propionic acid, butyric acid, valeric acid, non-metallic salts thereof, esters thereof and mixtures thereof, enabled effective treatment of dandruff and/or seborrheic dermatitis conditions associated with the proliferation of yeasts of the Malassezia genus without having an antimicrobial effect on Staphylococcus epidermidis and Cutibacterium acnes contrary to the medium chain fatty acids having a chain length comprising more than 6 carbon atoms as caproic acid, caprylic acid, ethylcaproate, glyceryl monocaprylate, propylene glycol monocaprylate, and metallic salt of short chain fatty acid as zinc propionate showed in the example 3 of the present application.

In addition to the selectivity on the Malassezia genus, the said short chain fatty acids mediate growth of Cutibacterium acnes, a commensal skin bacteria, which is decreased in dandruff and seborrheic dermatitis conditions.

Thus, these effects contribute to a rebalancing effect of the skin ecoflora.

A subject-matter of the present invention is therefore the cosmetic use of i) at least one short chain fatty acid chosen from propionic acid, butyric acid, valeric acid, non-metallic salts thereof, esters thereof and mixtures thereof, or of ii) a conditioned culture medium obtained from at least one microorganism which is able to produce one or more short chain fatty acid(s), the said medium comprising at least one short chain fatty acid chosen from propionic acid, butyric acid, valeric acid, non-metallic salts thereof, esters thereof and mixtures thereof, as an antidandruff agent.

Another subject-matter of the present invention is a cosmetic use of i) at least one short chain fatty acid chosen from propionic acid, butyric acid, valeric acid, non-metallic salts thereof, esters thereof and mixtures thereof, or of ii) a conditioned culture medium obtained from at least one microorganism which is able to produce one or more short chain fatty acid(s), the said medium comprising at least one short chain fatty acid chosen from propionic acid, butyric acid, valeric acid, non-metallic salts thereof, esters thereof and mixtures thereof, for preventing and/or treating desquamative disorders of the skin associated with the proliferation of yeasts of the Malassezia genus, more particularly of the Malassezia restricta species, such as dandruff and/or seborrheic dermatitis.

The invention also relates to a cosmetic use of i) at least one short chain fatty acid chosen from propionic acid, butyric acid, valeric acid, non-metallic salts thereof, esters thereof and mixtures thereof, or of ii) a conditioned culture medium obtained from at least one microorganism which is able to produce one or more short chain fatty acid(s), the said medium comprising at least one short chain fatty acid chosen from propionic acid, butyric acid, valeric acid, non-metallic salts thereof, esters thereof and mixtures thereof, for maintaining and/or restoring at a normal level, the ecoflora of the skin and especially by preventing excessive colonization of the skin by the yeasts of the Malassezia genus, more particularly Malassezia restricta species, and/or by mediating growth of Cutibacterium acnes.

Another subject-matter of the present invention is a cosmetic process intended for preventing and/or treating desquamative disorders of the skin associated with the proliferation of yeasts of the Malassezia genus, more particularly of the Malassezia restricta species, such as dandruff and/or seborrheic dermatitis, comprising the application, to the hair and/or skin, of a cosmetic composition comprising an effective amount of i) at least one short chain fatty acid chosen from propionic acid, butyric acid, valeric acid, non-metallic salts thereof, esters thereof and mixtures thereof, or ii) a conditioned culture medium obtained from at least one microorganism which is able to produce one or more short chain fatty acid(s), the said medium comprising at least one short chain fatty acid chosen from propionic acid, butyric acid, valeric acid, non-metallic salts thereof, esters thereof and mixtures thereof.

Another subject-matter of the present invention is a cosmetic process intended for maintaining and/or restoring at a normal level, the ecoflora of the skin and especially by preventing excessive colonization of the skin by the yeasts of the Malassezia genus, more particularly Malassezia restricta species, and/or by mediating the growth of Cutibacterium acnes, comprising the application, to the hair and/or skin, of a cosmetic composition comprising an effective amount of i) at least one short chain fatty acid chosen from propionic acid, butyric acid, valeric acid, non-metallic salts thereof, esters thereof and mixtures thereof, or ii) a conditioned culture medium obtained from at least one microorganism which is able to produce one or more short chain fatty acid(s), the said medium comprising at least one short chain fatty acid chosen from propionic acid, butyric acid, valeric acid, non-metallic salts thereof, esters thereof and mixtures thereof.

### Definitions

As used herein, the term “treating” or “treatment” refers to any action that to improve the comfort or the well-being of an individual. This term therefore covers attenuating, relieving or suppressing the symptoms of dandruff or seborrheic dermatitis, but is limited to a cosmetic treatment.

For the purposes of the present invention, the term “non-metallic salts” refers to salts which do not comprise metallic ion(s) such as, zinc ion, aluminium ion, copper ion, iron ion, and their mixtures.

For the purposes of the present invention, the term “skin” means the whole body skin, including scalp, preferably the skin of the scalp and facial skin such as forehead, nose, cheeks, chin, chest, neck.

As used herein, the term “skin ecoflora” means the microbial flora naturally present on the healthy skin, especially skin commensal microorganisms, as for example Staphylococcus epidermidis, and/or Staphylococcus capitis, and/or Cutibacterium acnes.

For the purposes of the present invention, the terms “preventing” means reducing the risk of manifestation of a phenomenon, especially in the context of the invention dandruff and seborrheic dermatitis.

For the purposes of the present invention, the terms “effective amount” means an amount that is sufficient to obtain the expected effect.

As used herein, the terms “cosmetic composition” means a composition suitable for an application on the skin, in particular a composition which comprises a physiologically acceptable medium.

The terms “physiologically acceptable medium” means a medium that is suitable for the topical administration of a composition, i.e. that is compatible with the skin of the face, the body and the scalp.

For the purposes of the present invention, the terms “short chain fatty acid” means a carboxylic acid with an aliphatic chain comprising 3 to 5 carbon atoms, preferably, a carboxylic acid with an aliphatic chain having 3 carbon atoms.

### DETAILED DESCRIPTION OF THE INVENTION

### Short Chain Fatty Acids (SCFA)

As stated previously, a subject-matter of the present invention is the cosmetic use of i) at least one short chain fatty acid chosen from propionic acid, butyric acid, valeric acid, non-metallic salts thereof, esters thereof and mixtures thereof, or of ii) a conditioned culture medium obtained from at least one microorganism which is able to produce one or more short chain fatty acid(s), the said medium comprising at least one short chain fatty acid chosen from propionic acid, butyric acid, valeric acid, non-metallic salts thereof, esters thereof and mixtures thereof, as an antidandruff agent.

The present invention also relates to the cosmetic use of i) at least one short chain fatty acid chosen from propionic acid, butyric acid, valeric acid, non-metallic salts thereof, esters thereof and mixtures thereof, or of ii) a conditioned culture medium obtained from at least one microorganism which is able to produce one or more short chain fatty acid(s), the said medium comprising at least one short chain fatty acid chosen from propionic acid, butyric acid, valeric acid, non-metallic salts thereof, esters thereof and mixtures thereof, for preventing and/or treating desquamative disorders of the skin associated with the proliferation of yeasts of the Malassezia genus, more particularly of the Malassezia restricta species, such as dandruff and/or seborrheic dermatitis.

The present invention also relates to the cosmetic of i) at least one short chain fatty acid chosen from propionic acid, butyric acid, valeric acid, non-metallic salts thereof, esters thereof and mixtures thereof, or of ii) a conditioned culture medium obtained from at least one microorganism which is able to produce one or more short chain fatty acid(s), the said medium comprising at least one short chain fatty acid chosen from propionic acid, butyric acid, valeric acid, non-metallic salts thereof, esters thereof and mixtures thereof, for maintaining and/or restoring at a normal level, the ecoflora of the skin and especially by preventing excessive colonization of the skin by the yeasts of the Malassezia genus, more particularly Malassezia restricta species and/or by mediating the growth of Cutibacterium acnes.

The non-metallic salts of short chain fatty acids according to the invention are particularly preferred and may be any safe and effective non-metallic salt of such acid. To illustrate, certain preferred salts may include calcium salts, sodium salts, magnesium salts, and potassium salts, the most particularly preferred being sodium salts.

As an additional illustration, amino acid salts may be utilized. For example, a carnitine or lysine salt of short chain fatty acids according to the invention may be utilized. The ordinarily skilled artisan will recognize that various other amino acids may be utilized as well.

As examples of esters of propionic acid, butyric acid, or valeric acid, any safe and effective ester of such acid may be utilized. For example, where the SCFA is an ester of propionic acid, the component can be represented as follows:

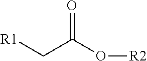

where R1=CH₃ and R2 is the ester chain or the ester of propionic acid.

As an example, the ester chain of the selected acid may be a straight or branched chain of carbon atoms and typically contains about 8 carbon atoms or less. This ester chain more preferably contains from 1 to about 5 carbon atoms and, again, may be a straight (for example, n-propyl) or branched (for example, iso-propyl) chain. Highly preferred ester chains include those that form methyl esters (i. e., R2 is —CH3), ethyl esters, n-propyl esters, iso-propyl esters, n-butyl esters, iso-butyl esters, and mixtures thereof. To illustrate, methyl propionate, ethyl propionate, n-propyl propionate, iso-propyl propionate, n-butyl propionate, iso-butyl propionate are examples of esters of propionic acid that may be used herein. Such esters of propionic, butyric or valeric acid may be selected as well.

As examples of short chain fatty acids, mention may be made of sodium propionate (Ref. P1880); Sodium butyrate (Ref. 303410); valeric acid (Ref. 75054) sold by Sigma.

In a particular embodiment, the short chain fatty acids are obtained from at least one microorganism which is able to produce one or more short chain fatty acid(s). The said at least one microorganism which is able to produce one or more short chain fatty acid(s) can be chosen from the group Lactobacillus spp, Bifidobacterium spp, Ruminococcus spp, Roseburia spp, Akkermansia muciniphila, Faecalibacterium spp, Eubacterium rectale, and Cutibacterium acnes, preferably Lactobacillus spp, Bifidobacterium spp, and Cutibacterium acnes, more preferably Cutibacterium acnes, such as the strain Cutibacterium acnes ATCC 6919.

In another embodiment, the short chain fatty acids according to the invention are contained in a conditioned culture medium (or supernatant) from at least one microorganism which is able to produce one or more short chain fatty acid(s).

The said at least one microorganism which is able to produce one or more short chain fatty acid(s) can be chosen from the group Lactobacillus spp, Bifidobacterium spp, Ruminococcus spp, Roseburia spp, Akkermansia muciniphila, Faecalibacterium spp, Eubacterium rectale, and Cutibacterium acnes, preferably Lactobacillus spp, Bifidobacterium spp, and Cutibacterium acnes, more preferably Cutibacterium acnes, such as the strain Cutibacterium acnes ATCC 6919.

A “culture supernatant” also called “conditioned culture medium” is typically obtained by culturing the microorganism concerned in a medium suitable for survival and/or the growth of the microorganism, then by separation of the medium and the microorganism so as to harvest the medium brought into contact with the microorganism. Preferably, the culture is carried out for a period of time and under conditions likely to allow the microorganism to release in the medium the active agents having the antidandruff properties desired, in particular the short chain fatty acids according to the invention.

The environment suitable for the survival and/or growth of the microorganism will be any nutrient media suitable for survival and/or culture of the microorganism. It usually contains a source of carbon and nitrogen, such as, for example, amino acids, sugars, proteins, fatty acids, phosphates, sulphates, minerals and growth factors and vitamins in adequate amounts.

For the purposes of the present application, the terms “conditioned culture medium” or “culture supernatant” are used indifferently to designate the entirety of the culture supernatant obtained after culture of the microorganism in question, or any fraction or sub-compound of the supernatant obtained by dialysis, fractionation, phase separation, filtration chromatography, affinity chromatography, precipitation, concentration, lyophilization, etc.

In the context of the present invention, conditioned culture medium from at least one microorganism which is able to produce one or more short chain fatty acid(s) according to the invention is obtained by the process comprising the following steps:
i) culturing at least one microorganism which is able to produce one or more short chain fatty acid(s), preferably microorganism of the species Cutibacterium acnes, such as Cutibacterium acnes ATCC 6919;
ii) separating, in particular by centrifugation, the culture supernatant from the biomass;
iii) recovering the culture supernatant; and
iv) optionally stabilizing the culture supernatant, for instance by filtration and/or autoclaving.

As used herein, the term “biomass” refers to the Cutibacterium acnes cells obtained after conducting the step i).

Preferably, the filtration is conducted with a syringe filter of pore size between 0.2 μm to 0.45 μm.

The short chain fatty acids or the conditioned culture medium according to the present invention is used in an amount representing from 0.01% to 5% by weight relative to the total weight of the composition, preferably in an amount representing from 0.3% to 1.0% by weight relative to the total weight of the composition.

Another subject-matter of the present invention is a cosmetic process intended for preventing and/or treating desquamative disorders of the skin associated with the proliferation of yeasts of the Malassezia genus, more particularly of the Malassezia restricta species, such as dandruff and/or seborrheic dermatitis, comprising the application, to the hair and/or skin, of a cosmetic composition comprising an effective amount of i) at least one short chain fatty acid chosen from propionic acid, butyric acid, valeric acid, non-metallic salts thereof, esters thereof and mixtures thereof, or ii) a conditioned culture medium obtained from at least one microorganism which is able to produce one or more short chain fatty acid(s), the said medium comprising at least one short chain fatty acid chosen from propionic acid, butyric acid, valeric acid, non-metallic salts thereof, esters thereof and mixtures thereof.

Another subject-matter of the present invention is a cosmetic process intended for maintaining and/or restoring at a normal level, the ecoflora of the skin and especially by preventing excessive colonization of the skin by the yeasts of the Malassezia genus, more particularly Malassezia restricta species, and/or by mediating the growth of Cutibacterium acnes, comprising the application, to the hair and/or skin, of a cosmetic composition comprising an effective amount of i) at least one short chain fatty acid chosen from propionic acid, butyric acid, valeric acid, non-metallic salts thereof, esters thereof and mixtures thereof, or ii) a conditioned culture medium obtained from at least one microorganism which is able to produce one or more short chain fatty acid(s), the said medium comprising at least one short chain fatty acid chosen from propionic acid, butyric acid, valeric acid, non-metallic salts thereof, esters thereof and mixtures thereof.

Preferably, said cosmetic composition comprises a cosmetically acceptable medium, that is to say a medium compatible with topical application to keratin materials, especially the skin.

Preferably, the pH of said cosmetic composition is between 6 and 8, notably between 6.5 and 7.5, and in particular at a neutral pH of 7.0.

Advantageously, the short chain fatty acid or the conditioned culture medium according to the present invention is present in an amount representing from 0.01% to 5% by weight relative to the total weight of the composition, preferably in an amount representing from 0.3% to 1% by weight relative to the total weight of the composition.

In a first preferred embodiment, said cosmetic composition is a composition for the scalp, which may be a rinse-out or a leave-on composition. The composition is preferably in the form of a shampoo, a cream, a mousse (aerosol or non-aerosol), a paste, a gel, an emulsion, a lotion or even a stick. Preferably, the composition for the hair is a shampoo, a gel or a lotion.

In second preferred embodiment, said cosmetic composition is a composition for the skin which may be more or less fluid and may have the appearance of a white or coloured cream, an ointment, a milk, a lotion, a serum, a paste or a foam. They may be optionally applied to the skin in aerosol form. They may also be in solid form, for example in the form of a stick or a compact powder. Especially, said cosmetic composition may especially be in the form of an aftershave gel or lotion, body hygiene composition such as a shower gel, a solid composition such as a soap or a cleansing bar, a composition for caring for or cleansing the skin.

The cosmetic composition preferably comprises water and/or one or more water miscible organic solvents that may be selected from linear or branched C1-C6 monoalcohols, such as ethanol, isopropanol, tert-butanol or n-butanol; polyols, such as glycerol, propylene glycol, hexylene glycol (or 2-methyl-2,4-pentanediol) and polyethylene glycols; polyol ethers, such as dipropylene glycol monomethyl ether; and mixtures thereof.

Preferably, the cosmetic composition comprises water in an amount ranging from 30 to 98% by weight, especially from 40 to 95% by weight, better from 50 to 90% by weight, relative to the total weight of the composition.

Preferably, the cosmetic composition comprises the organic solvent(s) in an amount ranging from 0.05 to 60%, preferably from 0.5 to 50% and better still from 1 to 40% by weight, relative to the total weight of the cosmetic composition.

Said cosmetic composition according to the invention may also comprise at least one customary cosmetic ingredient, especially selected from plant, mineral, animal or synthetic oils; liquid fatty alcohols; liquid fatty esters; solid fatty substances and notably waxes, solid fatty esters, solid alcohols; anionic, cationic, amphoteric and nonionic surfactants; anionic, nonionic, amphoteric and cationic polymers; antidandruff agents other than short chain fatty acid or the conditioned culture medium according to the present invention; antioxidants; agents for combating hair loss; silicones; perfumes; polymeric or non-polymeric thickeners, and notably associative polymers; optionally preservatives; chelating agent; colorants. The composition can, of course, comprise several cosmetic ingredients appearing in the above list. A person skilled in the art will take care to select the ingredients making up the composition, and also the amounts thereof, such that the advantageous properties of the composition according to the invention are not, or are not substantially, adversely affected by the envisaged addition.

The application of said short chain fatty acid or the conditioned culture medium or the composition to the hair and/or skin, can be followed, or not, by a rinsing step, for example with water.

Throughout the description, including the claims, the expression “comprising a” should be understood as being synonymous with “comprising at least one”, unless specified to the contrary.

In addition, the expression “at least one” should be understood as being synonymous with “one or more” unless specified to the contrary.

The expressions “more than”, “between . . . and . . . ” and “ranging from . . . to . . . ” must be understood with the limits included, unless the contrary is specified.

The examples and figures which follow are presented by way of illustration and without limitation of the invention.

The compounds are, as appropriate, cited in chemical names or in CTFA names (International Cosmetic Ingredient Dictionary and Handbook).

The invention is illustrated in more detail in the following examples.

### FIGURES

FIG. 1: Effect of SCFAs on Malassezia restricta growth.

FIG. 2: Effect of SCFAs on Cutibacterium acnes growth.

FIG. 3: Effect of SCFAs on Staphylococcus epidermidis growth.

FIG. 4: Effect of fatty acids (50 mM) on growth of Malassezia restricta, quantified using fluorescence staining.

FIG. 5: Effect of fatty acids esters and metal salts (50 mM) on growth of Malassezia restricta, quantified by measuring ATP.

FIG. 6: Effect of fatty acids, esters, and metal salts on growth of Cutibacterium acnes.

FIG. 7: Effect of fatty acids, esters, and metal salts on growth of Staphylococcus epidermidis.

FIG. 8: Effect of SCFAs on Cutibacterium acnes growth when Cutibacterium acnes is cultured in co-culture with Staphylococcus epidermidis in aerobic condition.

### EXAMPLES

### Example 1—Evaluation of the Selective Malassezia restricta Growth Inhibition by Sodium Propionate, Sodium Butyrate, and Sodium Valerate (According to the Invention)

A) Material and Methods

Organisms and Growth Conditions:

M. restricta ATCC MYA-4611, S. epidermidis ATCC 12228, and C. acnes ATCC 6919 were purchased from ATCC. M. restricta was routinely cultured in modified Dixon (MD) medium (pH 6), composed of 36 g of Malt extract (Sigma 70167), 20 g of Dessicated Oxbile (Sigma 70168), 6 g of Bacto™ Peptone (BD 211677), 1% (v/v) Tween 40 (Sigma P1504), 0.2% (v/v) Oleic Acid (Fluka 75096), and 0.2% (v/v) Glycerol (Promega H5433) in 1 liter of dH₂O. C. acnes and S. epidermidis were cultured in modified brain heart infusion (MBHI) medium (pH 7), containing 37 g of BHI base (Accumedia 7116B), 0.4% (v/v) of Tween-40 (Sigma P1504), 0.2% (v/v) of Oleic acid (Fluka 75096), and 0.2% (v/v) of Glycerol (Promega H5433) in 1 liter of dH2O. M. restricta and S. epidermidis were routinely grown under aerobic conditions, shaken at 200 rpm, and C. acnes was grown under anaerobic conditions. All organisms were grown at 33° C. Wherever required, media was supplemented with: sodium acetate (CH₃COOH, Sigma Ref. 32319), sodium propionate (CH3CH2COOH, Sigma Ref. P1880), sodium butyrate (CH₃(CH₂)₂COOH, Sigma Ref. 303410), valeric acid (CH₃(CH₂)₃COOH, Fluka Ref. 75054). For valeric acid, the pH in modified cell media was neutralized with NaOH (Sodium hydroxide, Sigma 283060).

Quantification of Total Cell Numbers by SYTO9:

M. restricta cells from 1 ml of the culture were harvested by centrifugation at 10,000×g for 5 minutes at room temperature. Cells were washed once, pelleted, and re-suspended in 0.9% NaCl solution. Equal volumes of the above cell suspension and SYTO 9 working stock (3 ul SYTO 9 component of the Live/Dead BacLight bacterial viability kit, ThermoFisher Scientific, L7012, diluted in 1 ml of 0.9% NaCl solution) were mixed well, incubated in dark for 10 minutes, and fluorescent intensity unit was measured in a Tecan microplate reader with excitation/emission wavelengths of 485/530 nm. A standard for correlation of fluorescence units and concentration was prepared using a fluorimeter and hemocytometer under a light microscope. This standard curve was used to calculate the total cell numbers.

Growth Assays:

M. restricta cells stored as glycerol stocks (30% glycerol in MD medium) were revived by plating on MD agar plates and incubated for 2-3 days at 33° C. A pre-culture was made from the lawn of cells grown on the agar plate. Cells from % of the plate was scrapped off, homogenized after suspending in 70 ml of MD medium in a 250 ml baffled erlenmeyer flask and incubated for 24 hours at 33° C. at 200 rpm. For experiments, 10⁷ cell/ml was inoculated in fresh MD medium with or without the short chain fatty acids. Growth was monitored by measuring the cell density with SYTO 9 for up to 24 hours or 96 hours. C. acnes and S. epidermidis were inoculated into MBHI medium at starting cell density of OD₆₀₀ 0.05 and 0.25, respectively. Bacterial growth was measured by plating the serially diluted cultures on Brain Heart Infusion agar plates at different time points and colonies were counted after 72 hours for C. acnes and 24 hours for S. epidermidis.

Quantification of Colony Forming Units (CFUs):

Bacterial growth was quantified by plating the serially diluted cultures on Brain Heart Infusion agar plates at different time points and colonies were counted after 72 hours for C. acnes and 24 hours for Staphylococcus sp.

B) Results

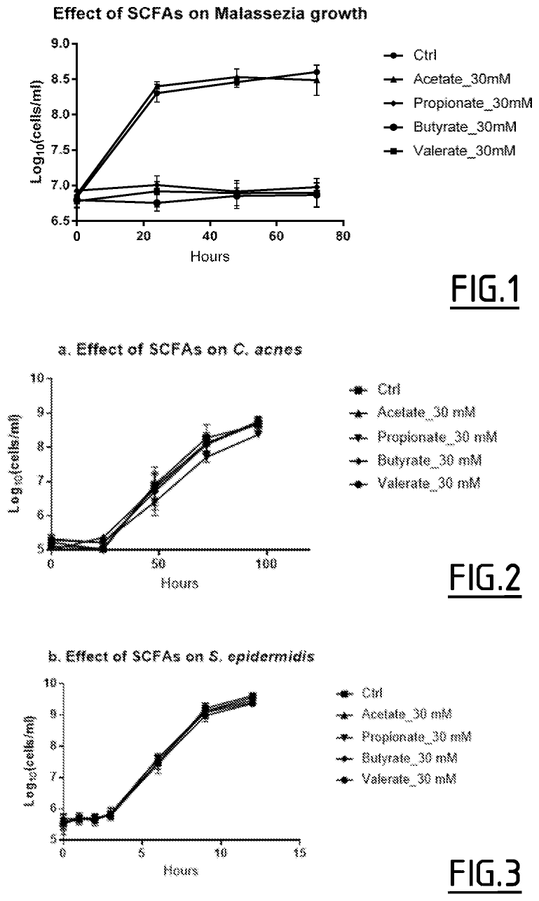

Sodium propionate, sodium butyrate, and sodium valerate completely inhibit the growth of Malassezia restricta at a concentration of 30 mM. No effect of acetate was seen at the same concentration (FIG. 1).

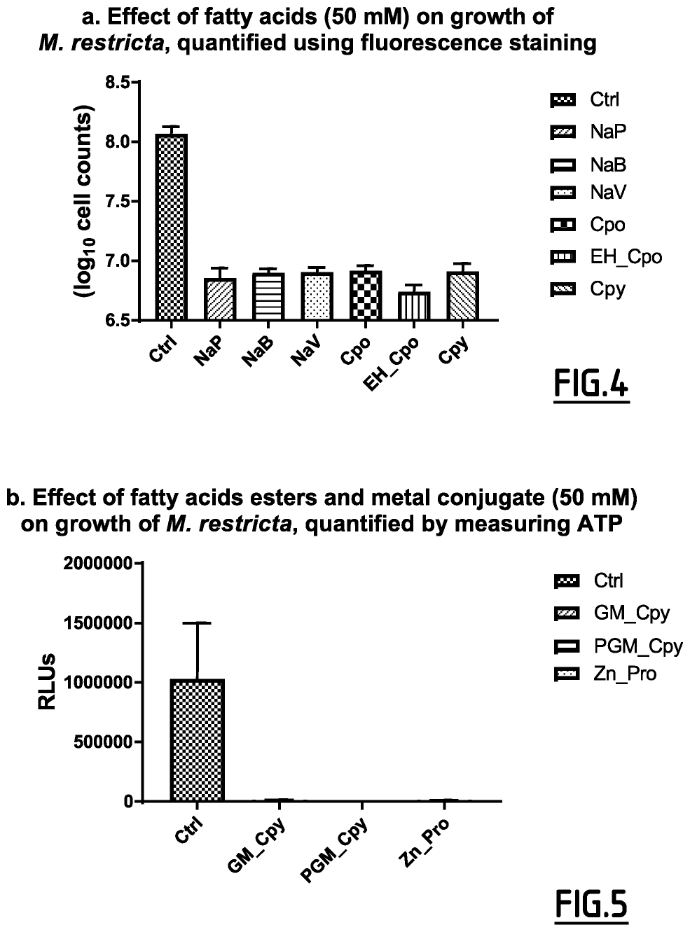

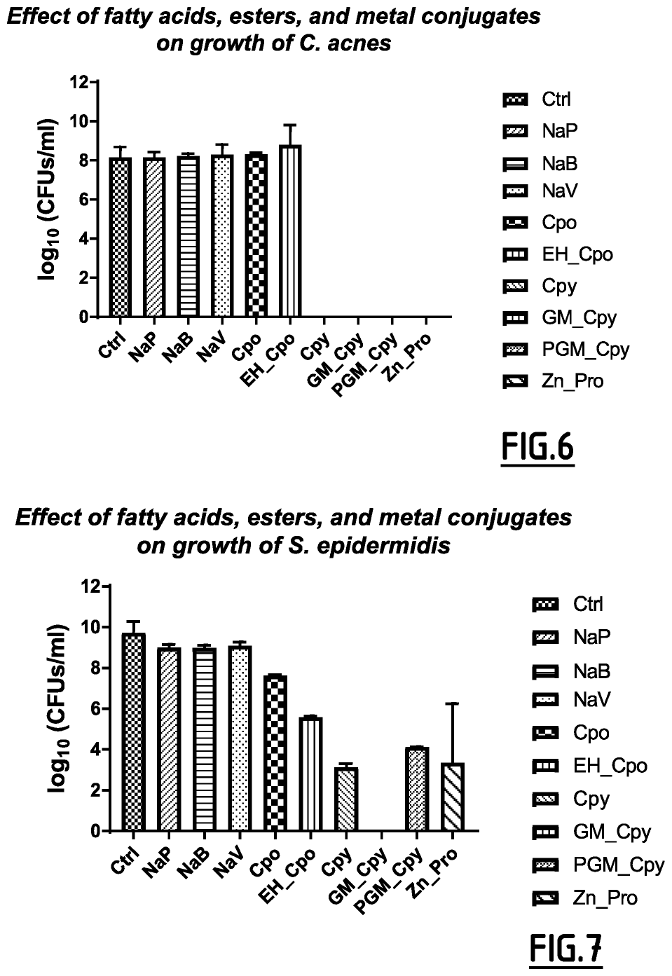

Sodium propionate, sodium butyrate, and sodium valerate do not affect the growth of C. acnes and S. epidermidis at concentrations where complete inhibition of Malassezia restricta was observed (30 mM) (FIGS. 2 and 3).

Conclusion: sodium propionate, sodium butyrate and sodium valerate selectively inhibit Malassezia restricta and do not affect the growth of the other major skin commensal microbes as C. acnes and S. epidermidis.

### Example 2—Evaluation of the Growth Promotion of Cutibacterium acnes by Sodium Propionate (According to the Invention)

A) Material and Methods

Organisms and Growth Conditions:

S. epidermidis ATCC 12228, and C. acnes ATCC 6919 were purchased from ATCC. C. acnes and S. epidermidis were cultured in modified brain heart infusion (MBHI) medium (pH 7), containing 37 g of BHI base (Accumedia 7116B), 0.4% (v/v) of Tween-40 (Sigma P1504), 0.2% (v/v) of Oleic acid (Fluka 75096), and 0.2% (v/v) of Glycerol (Promega H5433) in 1 liter of dH₂O. S. epidermidis was routinely grown under aerobic conditions, shaken at 200 rpm, and C. acnes was grown under anaerobic conditions. All organisms were grown at 33° C. Wherever required, media was supplemented with: sodium propionate (CH₃CH₂COOH, Sigma Ref. P1880),

Growth Assays:

C. acnes and S. epidermidis were inoculated in MBHI media at starting cell density of OD₆₀₀ 0.05 and 0.25 respectively. Bacterial growth was measured by plating the serially diluted cultures on Brain Heart Infusion agar plates at different time points and colonies were counted after 72 hours for C. acnes and 24 hours for S. epidermidis.

Co-Culture Assays:

For co-cultures, equal volumes of exponentially growing cells of the two species were mixed at cell density of 10⁵ cells per ml in 24-well glass bottom plates. Growth of each species in co-cultures was quantified by plating the culture suspension on agar medium. For selective quantification of each bacteria in the co-culture, agar plates were either grown under aerobic conditions to promote selective growth of S. epidermidis, or grown anaerobically to selectively enumerate C. acnes with furazolidone, which kills S. epidermidis alone.

Quantification of Colony Forming Units (CFUS):

Bacterial growth was quantified by plating the serially diluted cultures on Brain Heart Infusion agar plates at different time points and colonies were counted after 72 hours for C. acnes and 24 hours for Staphylococcus Sp.

B) Results

S. epidermidis was cultured until the formation of a biofilm for 24 hours to maximize its growth, and then C. acnes cells was added in oxygen saturated fresh media with or without propionate. C. acnes growth will be minimal unless propionate is metabolized by the S. epidermidis layer, which would create conditions for C. acnes to grow.

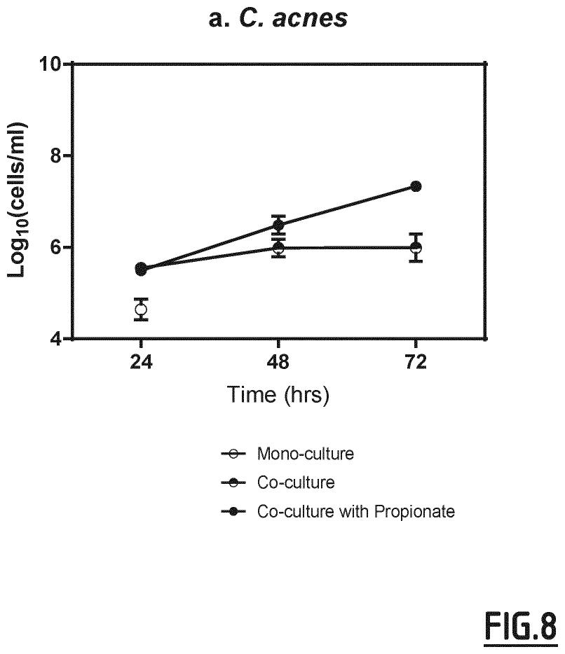

C. acnes and S. epidermidis growth was monitored for 24, 48 and 72 hours, and it has been found that C. acnes growth was augmented in presence of propionate (FIG. 8). The significantly higher growth of C. acnes under aerobic conditions in the presence of propionate demonstrates that propionate is able to modulate the community dynamics of the skin microbiome by promoting the growth of C. acnes in the presence of Staphylococcus Sp.

### Example 3—Evaluation of the Selectivity Malassezia restricta Growth Inhibition by Sodium Propionate, Sodium Butyrate, and Sodium Valerate (According to the Invention) Compared to Medium Chain Fatty Acid: Caproic Acid, Caprylic Acid, Ethyl Caproate, Glyceryl Monocaprylate, Propylene Glycol Monocaprylate (Outside of the Invention) and Metallic Salt of Short Chain Fatty Acid: Zinc Propionate (Outside of the Invention)

A) Material and Methods

Organisms and Growth Conditions:

M. restricta ATCC MYA-4611, S. epidermidis ATCC 12228 and C. acnes ATCC 6919 were purchased from ATCC. M. restricta were routinely cultured in Modified Dixon (MD) medium (pH 6), composed of 36 g of Malt extract (Sigma 70167), 20 g of Dessicated Oxbile (Sigma 70168), 6 g of Bacto™ Peptone (BD 211677), 1% (v/v) Tween 40 (Sigma P1504), 0.2% (v/v) Oleic Acid (Fluka 75096), and 0.2% (v/v) Glycerol (Promega H5433) in 1 liter of dH₂O. C. acnes and S. epidermidis were cultured in modified brain heart infusion (MBHI) medium (pH 7), containing 37 g of BHI base (Accumedia 7116B), 0.4% (v/v) of Tween-40 (Sigma P1504), 0.2% (v/v) of Oleic acid (Fluka 75096), and 0.2% (v/v) of Glycerol (Promega H5433) in 1 liter of dH2O. M. restricta and S. epidermidis were routinely grown under aerobic conditions, shaken at 200 rpm, and C. acnes was grown under anaerobic conditions. All organisms were grown at 33° C. Wherever required, media was supplemented with 50 mM of: sodium propionate (CH₃CH₂COOH, Sigma Ref. P1880), sodium butyrate (CH₃(CH₂)₂COOH, Sigma Ref. 303410), valeric acid (CH₃(CH₂)₃COOH, Fluka Ref. 75054). For valeric acid, the pH in modified cell media was neutralized with NaOH (Sodium hydroxide, Sigma 283060).

Quantification of Total Cell Number of M. restricta by SYTO9:

M. restricta cells from 1 ml of the culture were harvested by centrifugation at 10,000×g for 5 minutes at room temperature. Cells were washed once, pelleted, and re-suspended in 0.9% NaCl solution. Equal volumes of the above cell suspension and SYTO 9 working stock (3 ul SYTO 9 component of the Live/Dead BacLight bacterial viability kit, ThermoFisher Scientific, L7012, diluted in 1 ml of 0.9% NaCl solution) were mixed well, incubated in dark for 10 minutes and fluorescent intensity unit was measured in a Tecan microplate reader with excitation/emission wavelengths of 485/530 nm. A standard for correlation of fluorescence units and concentration was prepared using a fluorimeter and hemocytometer under a light microscope. This standard curve was used to calculate the total cell numbers.

Growth Assays:

M. restricta cells stored as glycerol stocks (30% glycerol in MD medium) were revived by plating on MD agar plates and incubated for 2-3 days at 33° C. A pre-culture was made from the lawn of cells grown on the agar plate. Cells from % of the plate was scrapped off, homogenized after suspending in 70 ml of MD medium in a 250 ml baffled erlenmeyer flask and incubated for 24 hours at 33° C. at 200 rpm. For experiments, 10⁷ cell/ml was inoculated in fresh MD medium with or without the short chain fatty acids at 50 mM concentration. Growth was monitored by measuring the cell density with SYTO 9 or by estimating the ATP concentration (see below) for up to 24 hours or 96 hours. C. acnes and S. epidermidis were inoculated in MBHI media at starting cell density of OD₆₀₀ 0.05 and 0.25, respectively. Bacterial growth was measured by plating the serially diluted cultures on Brain Heart Infusion agar plates at different time points and colonies were counted after 72 hours for C. acnes and 24 hours for S. epidermidis.

ATP Estimation:

Cellular ATP was measured using the BacTiterGlo Microbial cell viability (Promega) kit following manufacturers protocol. In short, Luciferase activity, which utilizes ATP as a substrate was estimated as a direct measure of ATP produced by the cells

B) Results

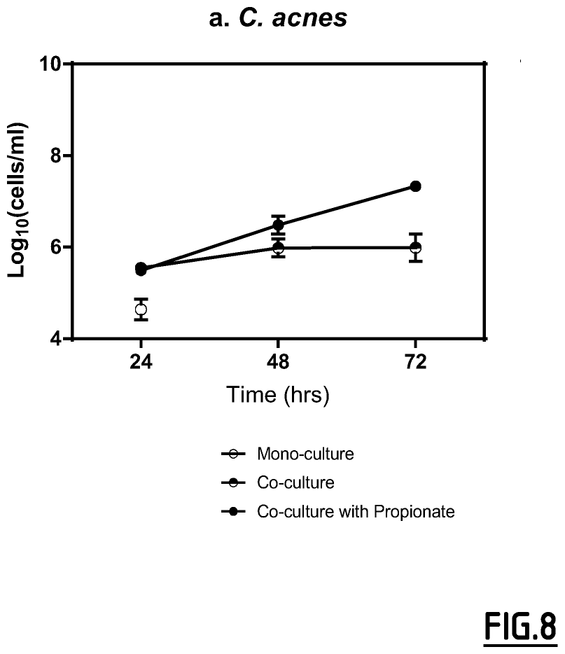

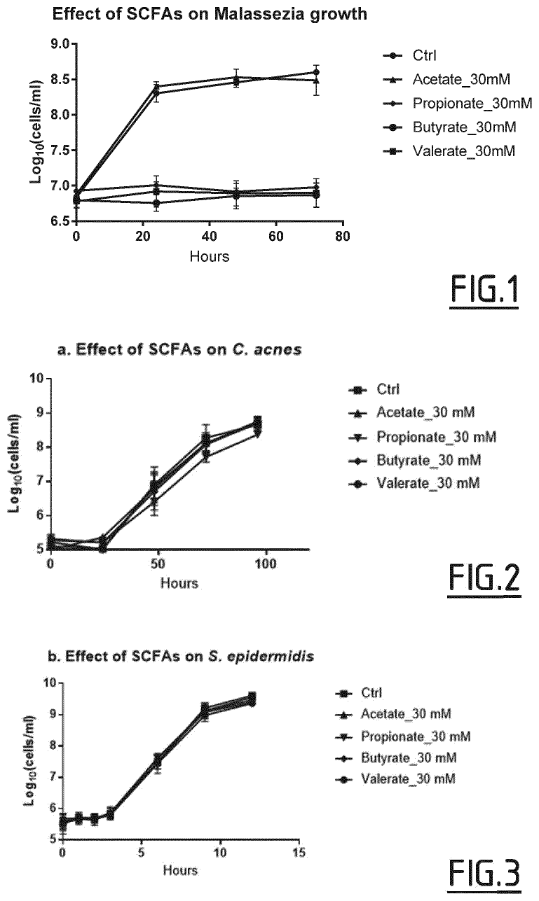

Due to varying characteristics of different compounds that interfered in the assay readouts, growth estimation of M. restricta was done using two different methods:
For all fatty acids and esters of caproic acid, total number of cells after 72 hours of incubation were quantified by fluorescence measurement of the cells stained with the fluorescent dye SYTO9 (see methods). The starting cell density of M. restricta was 1×10⁷ cells/ml (FIG. 4).
For esters of caprylic acid and Zinc propionate, cell viability was quantified by estimating the total amount of ATP in the cell lysate prepared from 24 hours grown cells (since these compounds made the medium opaque). ATP measurements were expressed in relative luminescence units (RLUs) of the luminescence emitted by the enzyme luciferase in the presence of ATP (FIG. 5).

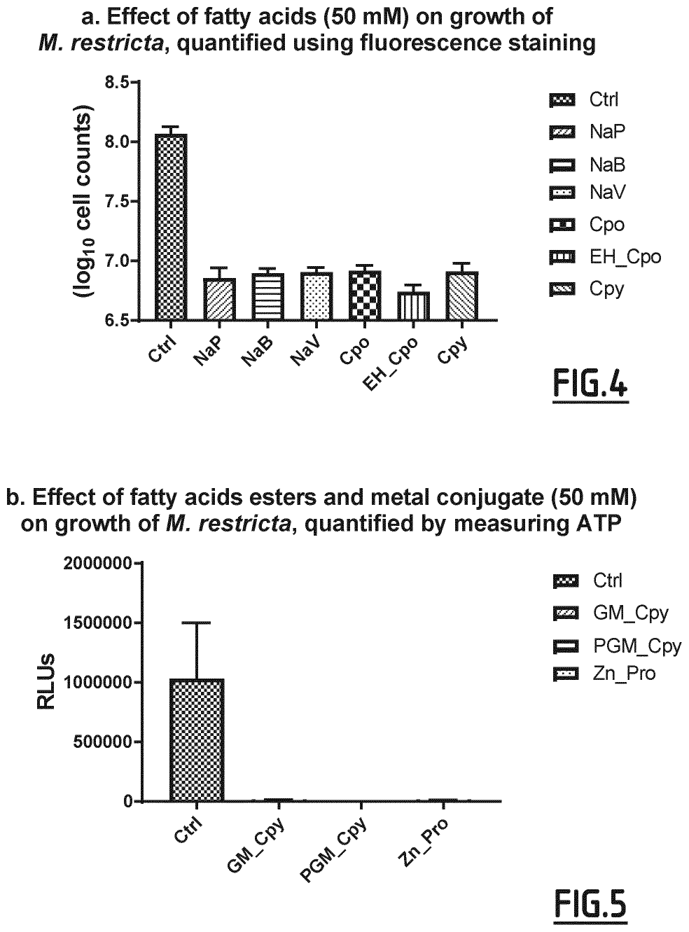

Growth of C. acnes was quantified by estimating the colony forming units (CFUs) after 72 hours of grown in media containing the fatty acids and their esters (FIG. 6).

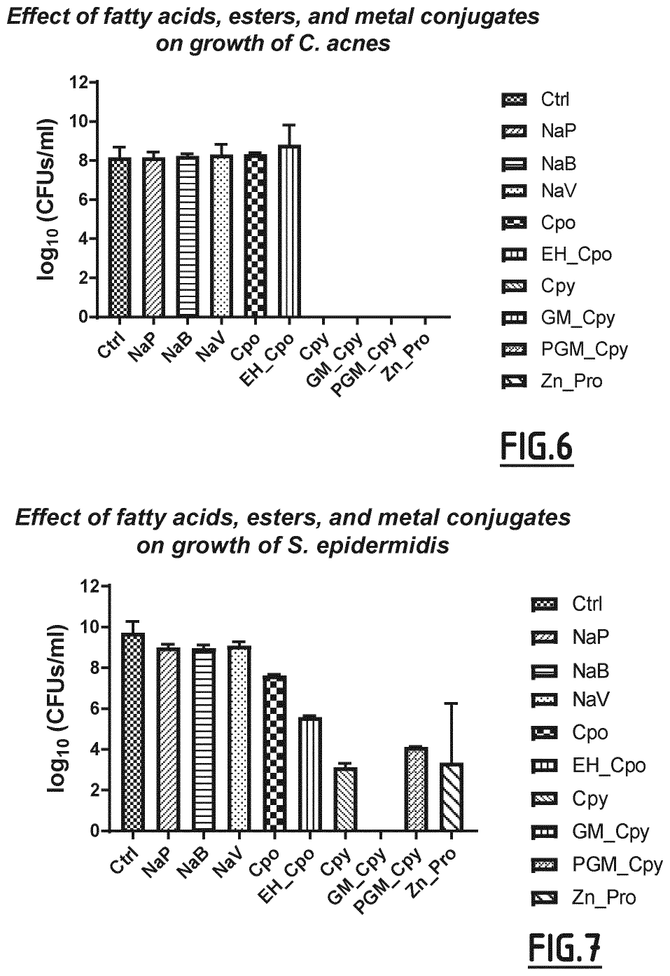

Growth of S. epidermidis was quantified by estimating the colony forming units (CFUs) after 24 hours of grown in media containing the fatty acids and their esters (FIG. 7). The results are presented in the tables 1 and 2 below.

TABLE 1
Tested compounds according to the invention

| | Rep. # | Ctrl | NaP | NaB | NaV |
|---|---|---|---|---|---|
| C. acnes | #1 | 8.37 | 7.82 | 8.26 | 8.90 |
| (log10 CFUs/ml) | #2 | 8.16 | 8.34 | 8.10 | 8.03 |
| | #3 | 8.05 | 8.29 | 8.34 | 8.00 |
| S. epidermidis | #1 | 9.55 | 8.82 | 8.87 | 9.29 |
| (log10 CFUs/ml) | #2 | 9.64 | 9.00 | 8.90 | 9.08 |
| | #3 | 8.72 | 9.16 | 9.15 | 8.90 |
| M. restricta | #1 | 8.06 | 6.95 | 6.94 | 6.86 |
| (log10 total cell counts) | #2 | 7.98 | 6.79 | 6.88 | 6.92 |
| | #3 | 8.01 | 6.82 | 6.88 | 6.93 |

*NaP: sodium propionate, NaB: sodium butyrate, NaV: sodium valerate.

TABLE 2
Tested compounds outside of the invention

| | Rep. # | Ctrl | Cpo | EH_Cpo | Cpy | GM_Cpy | PGM_Cpy | Zn_Pro |
|---|---|---|---|---|---|---|---|---|
| C. acnes | #1 | 8.37 | 8.29 | 9.94 | 0.00 | 0.00 | 0.00 | 0.00 |
| (log10 CFUs/ml) | #2 | 8.16 | 8.26 | 8.47 | 0.00 | 0.00 | 0.00 | 0.00 |
| | #3 | 8.05 | 8.41 | 8.00 | 0.00 | 0.00 | 0.00 | 0.00 |
| S. epidermidis | #1 | 9.55 | 7.67 | 5.54 | 3.03 | 0.00 | 4.15 | 5.05 |
| (log10 CFUs/ml) | #2 | 9.64 | 7.57 | 5.66 | 3.00 | 0.00 | 4.11 | 0.00 |
| | #3 | 8.72 | 7.64 | 5.55 | 3.34 | 0.00 | 4.12 | 5.00 |
| M. restricta | #1 | 8.06 | 6.93 | 6.80 | 6.98 | | | |
| (log10 total cell | #2 | 7.98 | 6.95 | 6.69 | 6.91 | | | |
| counts) | #3 | 8.01 | 6.87 | 6.74 | 6.84 | | | |
| (Relative | #1 | 543000 | | | | 16700 | −2410 | 12500 |
| luminescence | #2 | 1480000 | | | | −1470 | −2430 | 11900 |
| units) | #3 | 1070000 | | | | −2030 | −2270 | 10300 |

*Cpo: caproic acid, EH Cpo: ethyl caproate, Cpy: caprylic acid, GM Cpy: glyceryl mono caprylate, PGM Cpy: propylene glycol glyceryl mono caprylate, Zn Pr: zinc propionate.

Conclusion: sodium propionate, sodium butyrate and sodium valerate (compounds according to the invention) selectively inhibit M. restricta and do not affect the growth of the other major skin commensal microbes as C. acnes and S. epidermidis, in contrast to caproic acid, ethyl caproate, caprylic acid, glyceryl mono caprylate, propylene glycol glyceryl mono caprylate, zinc propionate (compounds outside of the invention) that were found to inhibit at least one of the skin commensals.

### Example 3—Facial Cream

The composition as described below is prepared.

TABLE 3
Facial cream composition

| Compounds | Concentration (w/w) |
|---|---|
| Sodium propionate | 1.0 |
| Sodium methyl stearoyl taurate | 0.23 |
| Xanthan gum | 0.05 |
| carbomer | 0.2 |
| water | qs 100 |

The composition is applied on the skin with a seborrheic dermatitis condition.

1-6. (canceled)

7. A cosmetic process intended for preventing and/or treating desquamative disorders of the skin associated with the proliferation of yeasts of the Malassezia genus, comprising the application, to the hair and/or skin, of a cosmetic composition comprising an effective amount of i) at least one short chain fatty acid chosen from propionic acid, butyric acid, valeric acid, non-metallic salts thereof, esters thereof and mixtures thereof, or ii) a conditioned culture medium obtained from at least one microorganism which is able to produce one or more short chain fatty acid(s), the said medium comprising at least one short chain fatty acid chosen from propionic acid, butyric acid, valeric acid, non-metallic salts thereof, esters thereof and mixtures thereof.

8. A cosmetic process intended for maintaining and/or restoring at a normal level, the ecoflora of the skin, comprising the application, to the hair and/or skin, of a cosmetic composition comprising an effective amount of i) at least one short chain fatty acid chosen from propionic acid, butyric acid, valeric acid, non-metallic salts thereof, esters thereof and mixtures thereof, or ii) a conditioned culture medium obtained from at least one microorganism which is able to produce one or more short chain fatty acid(s), the said medium comprising at least one short chain fatty acid chosen from propionic acid, butyric acid, valeric acid, non-metallic salts thereof, esters thereof and mixtures thereof.

9. The cosmetic process according to claim 7, wherein the short chain fatty acid or the conditioned culture medium is present in an amount representing from 0.01% to 5% by weight relative to the total weight of the composition.

10. The cosmetic process according to claim 7, wherein said cosmetic composition is in the form of a shampoo, a cream, a mousse, a paste, a gel, an emulsion, a lotion or a stick.

11. The cosmetic process according to claim 8, wherein the short chain fatty acid or the conditioned culture medium is present in an amount representing from 0.01% to 5% by weight relative to the total weight of the composition.

12. The cosmetic process according to claim 8 wherein said cosmetic composition is in the form of a shampoo, a cream, a mousse, a paste, a gel, an emulsion, a lotion or a stick.

13. The cosmetic process according to claim 7, wherein said desquamative disorder of the skin is dandruff and/or seborrheic dermatitis.

14. The cosmetic process according to claim 8, wherein said method prevents excessive colonization of the skin by the yeasts of the Malassezia genus and/or mediates growth of Cutibacterium acnes.

15. The cosmetic process according to claim 7, wherein the short chain fatty acid is chosen from sodium propionate, sodium butyrate, sodium valerate, and mixtures thereof.

16. The cosmetic process according to claim 7, wherein the short chain fatty acid is obtained from at least one microorganism of the species Cutibacterium acnes.

17. The cosmetic process according to claim 7, wherein the short chain fatty acid is obtained from at least one microorganism of the strain Cutibacterium acnes ATCC 6919.

18. The cosmetic process according to claim 7, wherein the conditioned culture medium is obtained by the process comprising the following steps:
a. culturing at least one microorganism which is able to produce one or more short chain fatty acid(s);
b. separating the culture supernatant from the biomass;
c. recovering the culture supernatant; and
d. optionally stabilizing the culture supernatant.

19. The cosmetic process according to claim 8, wherein the short chain fatty acid is chosen from sodium propionate, sodium butyrate, sodium valerate, and mixtures thereof.

20. The cosmetic process according to claim 8, wherein the short chain fatty acid is obtained from at least one microorganism of the species Cutibacterium acnes.

21. The cosmetic process according to claim 8, wherein the short chain fatty acid is obtained from at least one microorganism of the strain Cutibacterium acnes ATCC 6919.

22. The cosmetic process according to claim 8, wherein the said conditioned culture medium is obtained by the process comprising the following steps:
a. culturing at least one microorganism which is able to produce one or more short chain fatty acid(s);
b. separating the culture supernatant from the biomass;
c. recovering the culture supernatant; and
d. optionally stabilizing the culture supernatant.

23. The cosmetic process according to claim 22, wherein in step a. the microorganism is of the species Cutibacterium acnes; the separating in step b. is by centrifugation; and the stabilizing in step d. is by filtration and/or autoclaving.

24. The cosmetic process according to claim 23, wherein in step a. the microorganism is Cutibacterium acnes ATCC 6919.

25. The cosmetic process according to claim 18, wherein in step a. the microorganism is of the species Cutibacterium acnes; the separating in step b. is by centrifugation; and the stabilizing in step d. is by filtration and/or autoclaving.

26. The cosmetic process according to claim 25, wherein in step a. the microorganism is Cutibacterium acnes ATCC 6919.
